(19) United States
(12) Patent Application Publication
(10) Pub. No.: US 2003/0181389 A1
(43) Pub. Date: Sep. 25, 2003

(54) Therapeutic and prophylactic use of reduced forms of pharmaceutical compounds

(76) Inventors: Ernst Wülfert, (US); Anthony Atkinson, West Wellow (GB); Andres Marc Salomon, Salisbury (GB)

Correspondence Address: BAKER + HOSTETLER LLP, WASHINGTON SQUARE, SUITE 1100, 1050 CONNECTICUT AVE. N.W., WASHINGTON, DC, 20036-5304, US

(21) Appl. No.: 10/332,612

(22) PCT Filed: Jul. 10, 2001
(86) PCT No.: PCT/GB01/03081

(30) Foreign Application Priority Data
Jul. 11, 2000 (GB) 0017060.5

(51) Int. Cl. A61K 38/05; A61K 31/542; A61K 31/525; A61K 31/473; A61K 31/198; A61K 31/12

(52) U.S. Cl. 514/18; 514/224.8; 514/251; 514/298; 514/562; 514/690

(57) ABSTRACT

The reduced (or ‘leuco’) forms of certain pharmaceutically active compounds can be used for the treatment or pro-phylaxis methaemoglobinaemia or of a disease or disorder associated with or resulting from oxidative stress, such as Alzheimer's disease, motor neurone disease, Lewy Body disease, Pick's disease, Progressive Supranuclear Palsy, ischaemia, myocardial infarction, acute lung injury, stroke, Parkinson's disease or haemolysis and anaemia in acute falciparum malaria.

The present invention relates to the use of reduced forms of certain pharmaceutical compounds for the treatment and/or prophylaxis of various disorders, and specifically the treatment and prophylaxis of methaemoglobinaemia and of disorders arising from oxygen damage.

Methaemoglobin is an oxidation product of haemoglobin in which iron is in its ferric form (Fe3+), thus the molecule cannot bind oxygen reversibly. Ordinarily, one percent of haemoglobin is in this ferric state. Between 0.5 and three percent of deoxyhaemoglobin is normally spontaneously oxidised to methaemoglobin each day. The normal reducing power of erythrocytes maintains the balance between oxidation and reduction. NADH generated from glycolysis (the Embden Meyerhof pathway) acts as a substrate for a methaemoglobin reductase (NADH-cytochrome b5 reductase) enabling it to reduce methaemoglobin (Fe3+) back to haemoglobin. NADPH which is produced via the hexose monophosphate shunt serves as a substrate for another methaemoglobin reductase in methaemoglobin reduction (a fail-safe mechanism). In the hereditary enzymopenic form of methaemoglobinaemia, patients are homozygous or doubly heterozygous for a deficiency of NADH cytochrome b5 reductase. Consequently, erythrocytes of these patients contain excessive amounts of the oxidised (ferric) form of haemoglobin, which is incapable of oxygen transport.

Numerous drugs (such as dapsone, sulfsalazine, phenacetin, nitroglycerin, phenazopyridine hydrochloride, primaquine and vitamin K analogues) can insert themselves into the oxygen binding cleft of haemoglobin. By this action, such drugs can generate oxidised free radicals and peroxide. If the erythrocyte's protective reducing mechanisms are overwhelmed, haemoglobin is oxidised to forms of Heinz bodies and methaemoglobin, resulting in methaemoglobinaemia.

Patients with severe methaemoglobinaemia should be treated immediately with an intravenous solution of 1-2 mg/kg of methylene blue. In the presence of red cell enzyme NADPH-methaemoglobin reductase and adequate amounts of the electron donor NADPH, methylene blue is rapidly reduced in vivo to leucomethylene blue. This product in turn quickly reduces methaemoglobin to haemoglobin.

Reduction of methaemoglobin to haemoglobin by leucomethylene blue regenerates methylene blue. This cyclic reduction-oxidation process can go on as long as sufficient amounts of NADPH are produced by the pentose pathway in red blood cells. The success of methylene blue therapy therefore depends on the presence of adequate supplies of NADPH. Those patients who have abnormalities in the pentose phosphate pathway, such as G6 PD deficiency, will not respond to this approach and must receive emergency exchange blood transfusions.

G6 PD deficiency is one of the most common disorders in the world, approximately 10% of male blacks in the United States are affected, as are large numbers of black Africans and some inhabitants of the Mediterranean littoral. Consequently, significant numbers of subjects are at risk of (oxidative) drug-induced methaemoglobinaemia. Whilst the administration of methylene blue itself to such patients would be ineffective (because their G6 PD deficiency leads to an insufficiency of NADPH) and might even be counter-productive, administering stabilised leucomethylene blue to these patients should reduce methaemoglobin directly to haemoglobin and hence restore normal oxygen transport and delivery.

Repeated administration (in chronic toxicity studies) of high doses of methylene blue or thionine to laboratory animals causes oxidation of haemoglobin to methaemoglobin and tissue accumulation of Heinz bodies. These findings clearly indicate that both phenothiazines directly oxidise haem iron (to its ferric form) in vivo provided that the compounds are administered in sufficiently high amounts. The data also suggest that the toxicity of methylene blue may, at least in part, be related to its oxidation potential. The amount of NADPH produced in red blood cells is probably not sufficient to convert high dose levels of methylene blue to its corresponding (less toxic) leuco form.

A comparison of the redox potentials of thionine, Azure C and Azure B indicates that the ability of these dyes to be reduced, i.e. their capacity to attract electrons and hence act as oxidants, decreases with an increase in the number of methyl groups in their side chains (Murthy A and Reddy K S, J Chem Soc, Faraday Trans. 1984, 80: 2745-50). Thionine, which lacks methyl groups is therefore a more powerful oxidant than methylene blue.

Animal toxicity studies have shown that administration of high doses of thionine also causes oxidation of haemoglobin to methaemoglobin and accumulation of Heinz bodies. Since at least part of the toxicity of methylene blue and thionine is likely to be caused by oxidation of haem iron, one might infer from these redox studies that thionine would also be more toxic than methylene blue, which is indeed the case. Based on these observations, leucomethylene blue would therefore be expected to be considerably less toxic than its oxidised and highly ionised form, i.e. methylene blue.

Moreover, there is a growing body of evidence indicating that oxygen derived radicals (i.e. reactive oxygen species, “ROS”) directly cause tissue injury and cell death in numerous diseases. In particular, ROS have been convincingly shown to be causally involved in myocardial ischaemia-reperfusion injury occurring after an acute myocardial infarction, and in cerebral ischaemia-reperfusion injury occurring after acute ischaemic stroke. Oxidative stress is also thought to cause tissue injury and cell death in numerous other diseases, such as inflammatory bowel disease (IBD), rheumatoid arthritis (RA), glomerulonephritis, Alzheimer's disease (AD) and Parkinson's disease.

Oxygen superoxide anion and hydrogen peroxide have also been proposed as mediators of cyclosporin A (CsA)-induced nephrotoxicity, and treatment with antioxidants has been suggested in the prevention of CsA nephrotoxicity (Lopez-Ongil S. et al. Br J Pharmacol 1998, 124: 447-54; Parra T et al. Transplantation 1998, 66: 1325-29).

The extent of cell injury depends on the amount and type of radicals generated. Superoxide anion is a precursor of several more toxic species and their formation is accelerated upon reperfusion of ischaemic tissue. One therapeutic strategy is therefore based on the elimination of oxygen superoxide by antioxidants or by the administration of the enzyme superoxide dismutase, SOD.

Recent research has shown that stabilised leucomethylene blue is a powerful oxygen superoxide scavenger. This finding suggests that intervention therapy with stabilised leucomethylene blue should allow rapid elimination of tissue damaging oxygen superoxide radicals produced by ischaemia-reperfusion in conditions such as acute myocardial infarction, acute ischaemic stroke, and in acute post-ischaemic tubular necrosis (acute renal failure). Leucomethylene blue would also be expected to reduce CsA-induced nephrotoxicity and should therefore be administered concomitantly with cyclosporin A in conditions such as liver or kidney transplantation to prevent the nephrotoxicity commonly caused by and associated with CsA treatment.

Thus, the present invention provides the use of a reduced (leuco) form of a pharmaceutically active compound selected from the phenothiazines, riboflavin, the ubiquinones and 4,7-phenanthroline-5,-6-hydroquinone for the manufacture of a medicament for the treatment or prophylaxis of methaemoglobinaemia or of a disease or disorder associated with or resulting from oxidative stress.

The invention also provides the use of a reduced (leuco) form of a pharmaceutically active compound selected from the phenothiazines, riboflavin, the ubiquinones and 4,7-phenanthroline-5,6-hydroquinone for the manufacture of a medicament for the treatment or prophylaxis cyclosporin A induced nephrotoxicity.

Examples of the phenothiazines include Toluidine Blue 0 (tolonium chloride), Thionine, Azure A, Azure B, Azure C, Methylene Blue and 1,9-Dimethyl-methylene Blue. All of these compounds have in common the phenothiazine skeleton, and have a stable, but inactive, oxidised form and an active, but unstable, leuco form. Particularly preferred among these are methylene blue and thionine.

Other pharmaceutically active compounds which may used in the present invention include riboflavin, the ubiquinones, 4,7-phenanthroline-5, 6-hydroquinone and dapsone.

The present inventors have discovered a novel method for the conversion of a pharmaceutical compound from an oxidised form to a reduced form and/or for the stabilisation of that compound in a reduced state by admixing the oxidised form of the compound with ascorbic acid and with at least one sulphydryl compound. This invention forms the subject of a co-pending application filed on the same date as the present application.

We prefer that the pharmaceutically active compound used in the present invention should be stabilised by such a process.

The sulphydryl compound used in this stabilisation may be any compound having an —SR group, wherein S represents sulphur and R represents a hydrogen atom or a lower alkyl group, preferably having from 1 to 4, more preferably 1 or 2, carbon atoms. The —SH group is sometimes referred to as a ‘mercapto group’ and the two terms, ‘mercapto’ and ‘sulphydryl’, are sometimes used interchangeably. The stabilisation results in oxidation of the sulphydryl compound of the stabiliser, and it is preferred that the sulphydryl compound is such that the —H or —SR group is oxidised to a group of formula —S—S—. Preferred sulphydryl compounds are sulphur-containing amino acids and peptides, preferably oligopeptides, including at least one amino acid unit derived from such an amino acid, as well as derivatives of such amino acids and peptides, including salts, esters and amides thereof.

Preferred such amino acids include cysteine, methionine and ethionine. An example of a peptide including a unit derived from such an amino acid is glutathione. An example of a derivative (amide) of such an amino acid is N-acetylcysteine. Thus, preferred sulphydryl compounds are glutathione, cysteine, N-acetyl cysteine, methionine, ethionine, and mixtures of any two or more thereof.

The sulphydryl compound may be admixed with the pharmaceutically active compound before, after or simultaneously with the mixing of the pharmaceutically active compound with the ascorbic acid. The pharmaceutically active compound may alternatively be admixed with a composition containing ascorbic acid and at least one sulphydryl compound.

The ascorbic acid may be admixed with the pharmaceutically active compound in a weight ratio of from about 10:1 to about 100:1. The sulphydryl compound(s) may be mixed with the pharmaceutically active compound in a weight ratio of from about 2:1 to about 200:1. The weight ratio of the sulphydryl compound to ascorbic acid may be from about 1:0.5 to about 1:5.

The reduction may result in the conversion of some or all of the pharmaceutically active compound into a more reduced oxidation state. By way of example, more than 10 percent, more than 20 percent, more than 30 percent, more than 40 percent, more than 50 percent, more than 60 percent, more than 70 percent, more than 80 percent, more than 90 percent, or more than 95 percent of the pharmaceutically active compound may be converted into a more reduced form.

The oxidised form of the pharmaceutically active compound which is reduced in accordance with the invention may be present within a mixture or composition. The mixture or composition may comprise any of the known types of substance which are traditionally used in pharmaceutical compositions and medicaments.

Further substances may be admixed with the composition after the pharmaceutically active compound has been reduced. Examples of substances which may be added to the oxidised and/or reduced form of the pharmaceutically active compound are described elsewhere herein.

The pharmaceutically active compounds may be employed in the present invention alone or in admixture with various conventional additives to form a pharmaceutical composition. Additives include one or more pharmaceutically acceptable excipients, carriers, buffers, diluents, or preservatives.

A composition or medicament according to, produced by, or for use in the present invention preferably contains ascorbic acid and at least one sulphydryl compound in addition to the pharmaceutically active compound. The sulphydryl compound may be selected from the group consisting of glutathione, cysteine, N-acetylcysteine, methionine, ethionine, and mixtures thereof. The amount of ascorbic acid relative to the amount of the pharmaceutically active compound may be from about 10:1 to about 100:1 by weight. The amount of sulphydryl compound(s) may be from about 2:1 to about 200:1 by weight relative to the pharmaceutically active compound. The weight ratio of the sulphydryl compound to ascorbic acid may be from about 1:0.5 to about 1:5.

The pharmaceutically acceptable excipients, carriers, buffers, diluents and preservatives that may be mixed with the pharmaceutically active compound or composition containing it should ideally be non-toxic and should preferably not interfere with the activity of the pharmaceutically active compound. The precise nature of any excipient, carrier, buffer, diluent, preservative or other material within a composition or medicament may depend on the intended route of administration. Such materials are, however, well known to those skilled in the art and require no further explanation here.

A pharmaceutical composition or medicament of the invention that is ready for storage or administration may be in any suitable form, e.g. in the form of a tablet, capsule, powder, solution, suspension, or emulsion.

Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be used, alone or in combination with other carriers.

The pharmaceutical composition may be in the form of a parenterally acceptable aqueous solution which is pyrogen-free and has suitable pH and isotonicity. Those skilled in the art are well able to prepare suitable solutions using, for example, isotonic vehicles such as sodium chloride, Ringer's injection, or lactated Ringer's Injection.

Where the composition is in the form of a liquid, e.g. a solution, it may be degassed or sparged with an inert gas such as nitrogen or a noble gas (e.g. argon). Degassing or sparging may improve the stability of the reduced form of the pharmaceutical compound to re-oxidation. A liquid composition may be stored under an inert gas such as nitrogen or argon. It may be contained within an airtight biodegradable capsule which is suitable for administration. Where the composition is a tablet, the pharmaceutical compound may be reduced in solution. The tablet may be obtained by e.g. spray drying techniques which are well known to those skilled in the art. Such spray drying may occur under nitrogen or another inert gas in order to assist in maintaining the pharmaceutical compound in the reduced form. Tablets may be stored in airtight capsules, containers or packs (e.g. blister packs) to decrease their exposure to atmospheric oxygen. Such capsules, containers and packs are well known to those of skill in the art.

The subject may be an animal, particularly a mammal, which may be human or non-human, such as rabbit, guinea pig, rat, mouse or other rodent, cat, dog, pig, sheep, goat, cattle or horse, or which is a bird, such as a chicken.

Administration of the pharmaceutically active compound or composition is preferably in a “prophylactically effective amount” or a “therapeutically effective amount” as the case may be (although prophylaxis may be considered therapy) such an amount being sufficient to show benefit to the subject. The actual amount administered, and rate and time-course of administration, will depend on the nature and severity of what is being treated. Prescription of treatment, e.g. decisions on dosage etc, is within the responsibility of e.g. general practitioners and other medical doctors.

It is known that phenothiazines can be used in the modulation, e.g. inhibition, of tau-tau protein association and of neurofilament aggregation. In this regard, reference is made to International Published Patent Application No. WO 96130766, the entire disclosure of which is herein incorporated by reference. Modulation of tau-tau protein association and/or of neurofilament aggregation may be useful in the treatment of e.g. Alzheimer's disease, motor neurone disease, Lewy Body disease, Pick's disease and Progressive Supranuclear Palsy.

Until now there has been no recognition of whether the phenothiazines are active in an oxidised or in a reduced (“leuco”) form.

The disorder, disease or condition associated with or resulting from oxidative stress and to which the present invention relates may be selected from the group consisting of Parkinson's disease, Alzheimer's disease, motor neurone disease, Lewy Body disease, Pick's disease, Progressive Supranuclear Palsy and haemolysis and anaemia in acute falciparum malaria.

Without wishing to be bound by theory, it is believed that it is the leuco forms of the phenothiazines which are able to cross the blood-brain barrier. It is also believed that it is the oxidised (non-leuco) form of the phenothiazines that is responsible for the mutagenicity and toxicological problems that are observed in the literature.

Aspects of the invention which relate to the production, stabilisation and use of the reduced forms of the phenothiazines may therefore provide significant advances in treatments employing the phenothiazines, e.g. in the treatment of conditions, diseases or disorders which are associated with tau-tau association and/or neurofilament aggregation, e.g. Parkinson's disease, Alzheimer's disease, motor neurone disease, Lewy Body disease, Pick's disease and Progressive Supranuclear Palsy.

The present invention thus provides a method of treating methaemoglobinaemia, the method comprising the administration of a reduced form of a phenothiazine. The invention also provides for the use of a reduced form of a phenothiazine for the manufacture of a medicament for treating methaemoglobinaemia.

A further medical application of the reduced form of phenothiazines is the protection of tissues from oxidative damage.

Tissue damage associated with ischaemia and reperfusion injury results in Fe(V)O and Fe(V)0 states of haem proteins. These proteins then facilitate the production of cytotoxic oxygen radicals whose activity leads to oxidative damage.

Studies have shown that an amelioration or prevention of such oxidative damage can be effected by the administration of riboflavin. NADPH-dependent methaemoglobin reductase catalyses the intracellular reduction of riboflavin to dihydroriboflavin (Hultquist, D. E. et al (1993) Am. J. Hematol: January 1993; 42(1), p. 13 et seq). Dihydroriboflavin in turn reduces the Fe(IV)O and Fe(V)0 states of haem proteins, to prevent the formation of the radicals. Amelioration or prevention of oxidative damage associated with e.g. myocardial infarction, acute lung injury and stroke is possible.

Reduced phenothiazines such as leuco methylene blue present an alternative route to the reduction of the Fe(IV)0 and Fe(V)0 states of haem proteins. This route has only been made possible by the present invention providing the means to produce and stabilise the reduced form of these compounds.

The use of the phenothiazines in their reduced form has benefits in avoiding a dependence on NADPH and in reducing or preventing any toxicity associated with the oxidised compounds. The latter enables larger quantities of the compound to be administered.

Another instance in which oxidative tissue damage occurs is Parkinson's disease. There is evidence that oxygen superoxide is formed in Parkinson's disease and that the leuco forms of the phenothiazine compounds trap this reactive oxygen species, thereby preventing oxidative damage. The resulting decrease in the levels of neurotoxic reactive oxygen species formed in the Parkinsonian brain, thereby protects the dopaminergic neurones from the oxidative damage and neuronal death which contributes to the disease pathology.

It has also recently been shown that enhanced oxidative stress on erythrocytes in falciparum malaria may contribute substantially to haemolysis and anaemia [Das BS and Nanda NK, Trans R Soc Trop Med Hyg 1999,93(1): 58-62] and also that oxidative stress and erythrocyte damage may contribute to erythrocyte loss in children with severe Plasmodium falciparum malaria [Griffiths et al. Br J Haematol 2001, 113(2): 486-91], and so the administration of, for example, the reduced form of a phenothiazine, such as leucomethylene blue or leucothionine, could be expected to contribute to the treatment of these diseases.

In various further aspects, the present invention thus provides a method of ameliorating or preventing oxidative tissue damage, and a method of treating a disease, disorder or condition selected from the group consisting of ischaemia, myocardial infarction, acute lung injury, stroke, Parkinson's disease and haemolysis and anaemia in acute falciparum malaria. In each case, the methods comprise the administration of a reduced form of a phenothiazine.

The invention also provides for the use of a reduced form of a phenothiazine for the manufacture of a medicament for ameliorating or preventing oxidative tissue damage, and the use of a reduced form of a phenothiazine for the manufacture of a medicament for treating a disease, disorder or condition selected from the group consisting of ischaemia, myocardial infarction, acute lung injury, stroke, Parkinson's disease and haemolysis and anaemia in acute falciparum malaria.

The effectiveness of the leuco form of the phenothiazines in the treatment of diseases and disorders resulting from or associated with oxidative stress is illustrated by the following Examples. In these Examples, concentrated stocks of leucomethylene blue or leucothionine were stabilised in cysteine/ascorbic acid and were heavily diluted with nitrogen gassed buffers which had been blanketed with argon before use.

### EXAMPLE 1

Reduction of Ferricytochrome C

The principle upon which the following assay is based is that the enzyme xanthine oxidase acts on xanthine to oxidise xanthine and thereby to reduce ferricytochrome C by a mechanism which, at least partly, involves superoxide. Thus, the reduction (and increase) in optical density of ferricytochrome C is dependent on superoxide as a reductant. The inclusion of superoxide dismutase (“SOD”) in the assay serves to dismutate some of the available superoxide, thus causing a decrease in the rate of reduction of ferricytochrome C.

A SOD assay was performed in 50 mM potassium phosphate buffer (pH 7.8) horse heart ferricytochrome C (12.5 μM) and xanthine (50 μM) were mixed with EDTA (ethylenediamine tetraacetic acid) (100 μM) sufficient buttermilk xanthine oxidase (around 8 nm) was added to give a rate of increase in absorbance of 0.05-0.1 optical density units (550 nm) per minute at 30° C. (due to xanthine oxidation/cytochrome C reduction). Sufficient B. stearothermophilus SOD was then added to cause inhibition in the rate of the redox reaction by 25-75%.

Superoxide is a single electron reductant or oxidant. To prove that the oxidation of the leucomethylene blue and leucothionine is superoxide sensitive and, therefore, that both agents can act as superoxide scavengers, the following assay was set up. In both cases, the addition of SOD decreased the rate of oxidation of the leuco compounds, indicating that both leuco compounds were superoxide scavengers.

To explore the effect of leucothionine and leucomethylene blue, these compounds were mixed at 12.5 μM concentration in 50 mM potassium phosphate buffer (pH 7.8) with xanthine (50 μM) and EDTA (100 μM) buttermilk xanthine oxidase (around 10 nm) was then added to give a rate of increase in absorbance of about 0.1 optical density units (for methylene blue at 665 nm and for thionine at 605 nm) per minute at 30° C. above controls using leucothionine or leucomethylene blue, potassium phosphate buffer, xanthine and EDTA alone.

B. stearothermophilus SOD was then added to the reaction cell after the sixth ute to see if it reduced the rate of increase in absorbance in order to prove that the dation of the leuco compounds was due to superoxide. In both cases, the rate of rease was significantly reduced by the addition of 5 units of SOD. The results are shown in the following Table 1.

TABLE 1

| | control reduction of | oxidation of | oxidation of |
|---|---|---|---|
| Time | ferricytochrome C | leucomethylene blue | leucothionine |
| (minutes) | (550 μM) | (665 nm) | (605 nm) |
| 1 | 0.072* | 0.096 | 0.086 |
| 2 | 0.075 | 0.102 | 0.093 |
| 3 | 0.074 | 0.110 | 0.093 |
| 4 | 0.068 | 0.101 | 0.095 |
| 5 | 0.071 | 0.099 | 0.101 |
| 6 | 0.074 | 0.103 | 0.096 |
| 7 | | 0.030 * | 0.041 * |
| 8 | | 0.017 | 0.009 |
| 9 | | 0.014 | 0.009 |
| 10 | | 0.013 | 0.011 |
| 11 | | 0.010 | 0.008 |
| 12 | | 0.010 | 0.009 |

*5 units of SOD were added immediately after the sixth minute.

The difference between the rates of change of optical density shown in the above Table before and after the addition of the SOD immediately after the sixth minute is striking and clearly demonstrates the activity of the compounds of the present invention.

### EXAMPLE 2

In a further experiment, potassium superoxide was added to a reaction cell containing buffers, EDTA and concentrations of leucothionine and leucomethylene blue. Direct oxidation of the leuco compounds was observed by superoxide.

Potassium superoxide was added directly to nitrogen gassed potassium phosphate buffer (pH 7.8) containing EDTA (100 μM) and approximately 10 μM concentration of leucothionine and leucomethylene blue. In both cases, vigorous reactions occurred and the leuco compounds were rapidly oxidised to their coloured oxidised states.

Due to the effervescence of the cell, a known property of potassium superoxide in water, no measurements were possible in the first 4 minutes of the reaction. However, in both cases, the cell was observed to go from colourless to blue (leucomethylene blue) or purple (leucothionine). The results, in terms of the final optical densities, are shown in the following Table 2.

TABLE 2

| | final optical density | |
|---|---|---|
| | leucomethylene blue | final optical density |
| Time (minutes) | (665 nm) | leucothionine (605 nm) |
| 0 | 0.015 | 0.035 |
| 1* | | |
| 5 | 0.38 | 0.42 |
| 6 | 0.44 | 0.45 |
| 7 | 0.46 | 0.45 |
| 8 | 0.44 | 0.46 |

*Due to the effervescence of the cell, no measurements were possible in the first 4 minutes of the reaction.

### EXAMPLE 3

In another experiment, the results of the experiment in Example 1 above were confirmed by using the adrenochrome system. In this system, dl-epinephrine is auto-oxidised under alkaline conditions by a superoxide dependent pathway. The reaction rate of this reaction can be decreased or interrupted by the addition of SOD, which scavenges superoxide. The reaction rate can also be decreased or interrupted by the addition of leucomethylene blue or leucothionine, indicating that they are also superoxide scavengers. In this system, the rate of auto-oxidation of dl-epinephrine is decreased by the presence of either leuco compound, while the leuco compounds themselves are oxidised by superoxide in a rate dependent manner.

In this experiment, dl-epinephrine (500 μM) was allowed to auto-oxidise in 50 mM sodium carbonate buffer (pH 10.2) containing EDTA (100 μM) at 30° C. under these conditions, epinephrine is oxidised to adrenochrome (310 or 485 nm increase in absorption). In both cases, leucomethylene blue (50 μM) and leucothionine (50 μM) were effective in reducing the appearance of adrenochrome at 310 nm while they themselves were oxidised, as shown by an increasing oxidation peak at 665 nm and 605 nm, respectively.

The results are shown in the following Table 3.

TABLE 3

| | Epinephrine oxidation | | Leucomethylene blue | | Leucothionine | |
|---|---|---|---|---|---|---|
| Time (minutes) | (310 nm) | (485 nm) | (310 nm) | (665 nm) | (310 nm) | (605 nm) |
| 0 | 0.001 | 0.002 | 0.005 | 0.006 | 0.005 | 0.007 |
| 1 | 0.14 | 0.047 | 0.031 | 0.093 | 0.034 | 0.083 |
| 2 | 0.14 | 0.048 | 0.021 | 0.110 | 0.027 | 0.121 |
| 3 | 0.15 | 0.048 | 0.030 | 0.120 | 0.033 | 0.110 |
| 4 | 0.14 | 0.048 | 0.035 | 0.116 | 0.034 | 0.114 |
| 5 | 0.15 | 0.043 | 0.031 | 0.121 | 0.028 | 0.116 |
| 6 | 0.14 | 0.049 | 0.038 | 0.119 | 0.032 | 0.120 |

1. The use of a reduced form of a pharmaceutically active compound selected from the phenothiazines, riboflavin, the ubiquinones, 4,7-phenanthroline-5,6-hydroquinone and dapsone for the manufacture of a medicament for the treatment or prophylaxis of methaemoglobinaemia or of a disease or disorder associated with or resulting from oxidative stress or for the treatment or prophylaxis cyclosporin A induced nephrotoxicity.

2. The use according to claim 1, in which the reduced form is stabilised by admixture with ascorbic acid and with at least one sulphydryl compound.

3. The use according to claim 2, in which the sulphydryl compound is an sulphur-containing amino acid or a peptide including at least one amino acid unit derived from such an amino acid, or a derivative of such an amino acid or peptide.

4. The use according to claim 3, in which said derivative is a salt, ester or amide.

5. The use according to claim 3 or 4, in which said amino acid is cysteine or methionine.

6. The use according to claim 2, in which the sulphydryl compound is glutathione, cysteine, N-acetyl-cysteine, methionine, or a mixture of any two or more thereof.

7. The use according to any one of the preceding claims, in which the pharmaceutically active compound is a phenothiazine.

8. The use according to claim 7, in which the phenothiazine is Toluidine Blue C, Thionine, Azure A, Azure B, Azure C, Methylene Blue or 1,9-Dimethyl-methylene Blue, or a mixture of any two or more thereof.

9. The use according to claim 8, in which the pharmaceutical compound is methylene blue or thionine.

10. The use according to any one of claims 2 to 9, in which the weight ratio of ascorbic acid to the pharmaceutically active compound is from about 10:1 to about 100:1.

11. The use according to any one of claims 2 to 10, in which the weight ratio of sulphydryl compound(s) to the pharmaceutically active compound is from about 2:1 to about 200:1.

12. The use according to any one of claims 2 to 11, in which the weight ratio of sulphydryl compound to ascorbic acid is from about 1:0.5 to about 1:5.

13. The use according to any one of the preceding claims, for the manufacture of a medicament for the treatment or prophylaxis of Alheimer's disease, motor neurone disease, Lewy Body disease, Pick's disease or Progressive Supranuclear Palsy.

14. The use according to any one of claims 1 to 12, for the manufacture of a medicament for the amelioration or prevention of oxidative tissue damage.

15. The use according to claim 14, wherein the oxidative tissue damage is associated with ischaemia, myocardial infarction, acute lung injury, stroke or Parkinson's disease.

16. The use according to any one of the preceding claims, for the manufacture of a medicament for the treatment or prophylaxis of methaemoglobinaemia.

17. The use according to any one of the preceding claims, for the manufacture of a medicament for the treatment or prophylaxis of haemolysis and anaemia in acute falciparum malaria.
